(19) United States
(12) Patent Application Publication
(10) Pub. No.: US 2010/0312131 A1
(43) Pub. Date: Dec. 9, 2010

(54) NOISE DETECTION AND RESPONSE FOR USE WHEN MONITORING FOR ARRHYTHMIAS

(76) Inventors: Mihir Naware, San Jose, CA (US); Cecilia Qin Xi, San Jose, CA (US)

Correspondence Address: STEVEN M MITCHELL;PACESETTER INC, 701 EAST EVELYN AVENUE, SUNNYVALE, CA, 94086, US

(21) Appl. No.: 12/481,434

(22) Filed: Jun. 9, 2009

(51) Int. Cl. A61B 5/046

(52) U.S. Cl. 600/518

(57) ABSTRACT

Methods and systems of noise detection and response for use when monitoring for arrhythmias are described herein. At least two electrodes are used to obtain a signal indicative of cardiac electrical activity. The signal is bandpass filtered to obtain a filtered signal. Ventricular depolarizations are monitored for based on comparisons of the filtered signal to a first threshold. Arrhythmias are monitored for based on ventricular depolarization detections that occur as a result of monitoring for ventricular depolarizations. During one or more noise detection windows, noise is monitored for and a likelihood that monitoring for arrhythmias is adversely affected by noise is determined based on results thereof. Whether and/or how the monitoring for arrhythmias is performed is modified when it is determined that monitoring for arrhythmias is likely adversely affected by noise.

### FIELD OF THE INVENTION

Embodiments of the present invention relate to methods, systems and devices that can detect noise and respond to noise detections when monitoring for arrhythmias.

### BACKGROUND OF THE INVENTION

It can be desirable to monitor cardiac activity using a dedicated cardiac monitor that does not necessarily provide therapeutic response to arrhythmic episodes. For example, a cardiac monitor can be implanted in a patient with recurrent unexplained syncope to allow a physician to obtain a symptom-rhythm correlation during infrequent spontaneous symptoms such as syncope or pre-syncope. An electrocardiogram (ECG, or alternatively EKG) can be obtained by a dedicated cardiac monitor that is implanted subcutaneously without leads and electrodes positioned directly within or on the heart, as is required with implantable cardioverter-defibrillators (ICDs). Such devices enable use of minimally invasive surgical techniques for implantation to obtain valuable information to help a physician diagnose the causes of symptoms, such as syncopal episodes, and provide appropriate patient care.

Cardiac monitors implanted subcutaneously can be susceptible to baseline wander, environmental noise, and physiological noise. Examples of noise sources include respiration, muscle activity, power line interference, and electronic article surveillance (EAS) interference. Severe noise levels may result in inappropriate QRS detection, which can lead to false detections of arrhythmic episodes such as ventricular tachycardia (VT) and/or atrial fibrillation (AF) episodes. Standard noise detection algorithms, such as are implemented with heart rate measurements obtained from ICDs, use discrete detection windows to detect noise generally associated with electrical power line interference at 50/60 Hz. Such standard noise detection algorithms are not effective to detect and filter noises from such noise sources as myopotential, which signals are emitted with a frequency typically in the 20-80 Hz range.

Cardiac monitors implanted within or on the heart, such as ICDs, are also susceptible to noise; however, problematic noise may more likely be associated with defective components of the cardiac monitors, such as fractured leads, than physiological or environmental noise sources. Standard noise detection algorithms are not effective to detect and filter noise from fractured leads, for example, some part of which is in the 15-20 Hz range.

### SUMMARY OF THE INVENTION

Embodiments of the present invention are related to systems, devices, and methods for use therewith for detecting and responding to noise when monitoring for arrhythmias. In accordance with an embodiment, an implanted device having or connected to at least two electrodes is used to obtain a signal indicative of cardiac activity (e.g. an electrocardiogram (ECG)), which is bandpass filtered to obtain a filtered signal. The implanted device can be subcutaneously implanted, but is not limited thereto. In an alternative embodiment, the device can be a non-implanted device including or connected to surface electrodes useful for obtaining a surface ECG. In further embodiments, the device can be an implantable cardioverter-defibrillator (ICD) and/or pacemaker and the signal can be an intracardiac electrogram (IEGM) signal obtained using electrodes implanted within or on the patient's heart. The filtered signal, which in an embodiment has an effective frequency range of about 8.5 Hz to about 30 Hz, is monitored for ventricular depolarizations based on comparisons to a primary threshold. Arrhythmias are monitored for based on ventricular depolarization detection frequency and proximity. Noise is monitored for during one or more recurring noise detection windows to determine whether arrhythmia monitoring is likely adversely affected by noise. In a preferred embodiment, noise detection windows recur continuously without overlap or gaps between windows, i.e. the noise detection windows recur back-to-back. However, in alternative embodiments, the noise detection windows can overlap or alternatively be separated by a small gap. When monitoring for arrhythmias is determined to be likely adversely affected by noise, whether and/or how monitoring for arrhythmias is performed is modified.

Noise can be monitored by comparing portions of the filtered signal corresponding to one or more noise detection windows to a noise threshold having a lower magnitude than the primary threshold. In an embodiment, the noise threshold can be a specified percentage of the primary threshold, for example between 25% and 85%, and the recurring noise detection windows can span a length of time having a range between 90 ms and 250 ms. Noise is determined to be detected when the filtered signal crosses the noise threshold a designated number of times within a noise detection window. The designated number of threshold crossings can be directly related to the frequency of the noise signal being monitored for. For example, four or more threshold crossings may correspond to a noise frequency of 20 Hz and higher, while eight or more threshold crossings may correspond to a noise frequency of 40 Hz and higher, In an embodiment, the designated number of threshold crossings is four or more.

Monitoring for arrhythmias can be determined to be likely adversely affected by noise if noise detections occur for a specified number of preceding intervals between consecutive ventricular depolarizations (e.g., R-to-R intervals). In an embodiment, if noise detections occur for X out of the last Y (e.g., 3 out of the last 5) preceding R-to-R intervals, where Y is an integer ≧2, and X is an integer ≦Y, then monitoring for arrhythmias is determined to be likely adversely affected by noise, and noise mode is entered. Noise mode can comprise inhibiting monitoring for arrhythmias, ignoring arrhythmia detections, or storing information about arrhythmia detections along with an indication that the arrhythmia detections occurred when it was determined that monitoring for arrhythmias was likely adversely affected by noise.

This description is not intended to be a complete description of, or limit the scope of, the invention. Other features, aspects, and objects of the various embodiments of the present invention can be obtained from a review of the specification, the figures, and the claims.

### BRIEF DESCRIPTION OF THE FIGURES

FIGS. 1A and 1B illustrate implantable cardiac monitoring devices that include subcutanteous electrodes, according to specific embodiments of the present invention.

FIG. 1C is used to illustrate an implantable cardiac monitoring device, according to an embodiment of the present invention, implanted subcutaneously in a patient's pectoral region.

FIG. 1D is used to illustrate an implantable cardiac monitoring device, according to an embodiment of the present invention, that includes subcutaneous electrodes that are remote from the device housing.

FIG. 2A illustrates sample electrocardiograms (ECGs) recorded for a person engaging in isometric activity between periods of rest.

FIG. 2B is a schematic representation of one cycle of a typical ECG.

FIG. 3 is a flow chart of a method according to an embodiment of the present invention of detecting and responding to noise within an ECG which may otherwise be indicative of an arrhythmia.

FIG. 4 is a detailed flow chart of a method according to an embodiment of the present invention of detecting and responding to noise within an ECG which may otherwise be indicative of an arrhythmia.

FIG. 5 illustrates sample ECGs recorded for a person engaging in isometric activity between periods of rest including noise markers to identify entry and exit of noise mode, according to an embodiment of the present invention.

FIG. 6 illustrates a sample screenshot including an ECG as displayed in an embodiment of a device for use with systems and methods in accordance with the present invention.

FIG. 7 illustrates an exemplary subcutaneously implantable cardiac device that includes an arrhythmic event monitor which can be used to perform embodiments of the present invention.

FIG. 8A illustrates an exemplary implantable stimulation device that includes leads positioned within structures related to the heart, and which can be used to perform embodiments of the present invention.

FIG. 8B is a simplified block diagram that illustrates possible components of the implant device shown in FIG. 8A.

### DETAILED DESCRIPTION OF THE INVENTION

The following description is of the best modes presently contemplated for practicing various embodiments of the present invention. The description is not to be taken in a limiting sense but is made merely for the purpose of describing the general principles of the invention. The scope of the invention should be ascertained with reference to the claims. In the description of the invention that follows, like numerals or reference designators will be used to refer to like parts or elements throughout. In addition, the first digit of a reference number identifies the drawing in which the reference number first appears.

It would be apparent to one of skill in the art that the present invention, as described below, may be implemented in many different embodiments of hardware, software, firmware, and/or the entities illustrated in the figures. Any actual software, firmware and/or hardware described herein is not limiting of the present invention. Thus, the operation and behavior of the present invention will be described with the understanding that modifications and variations of the embodiments are possible, given the level of detail presented herein.

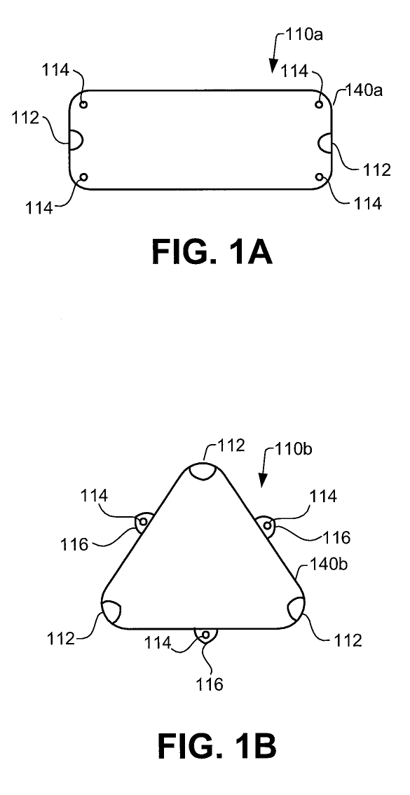

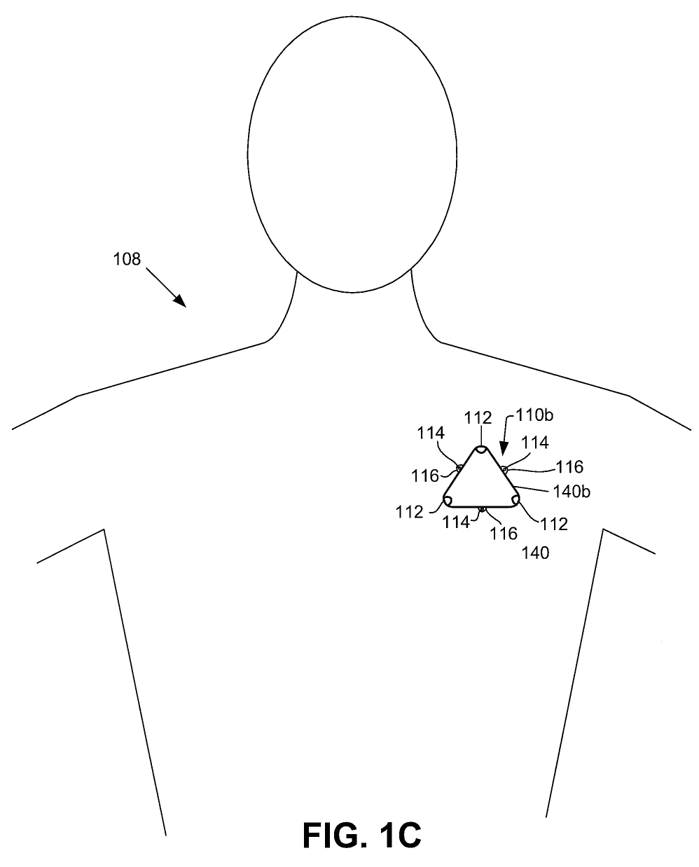

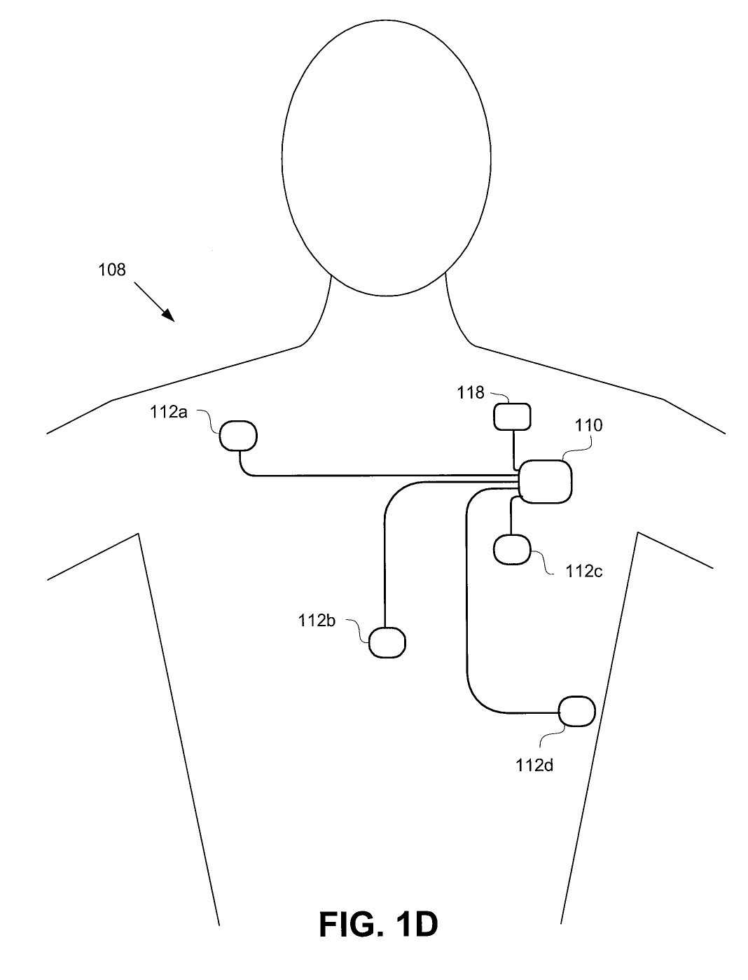

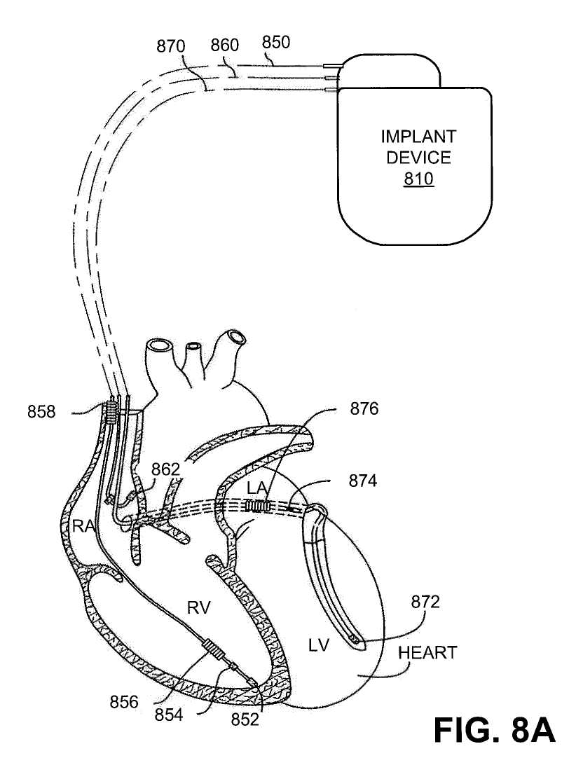

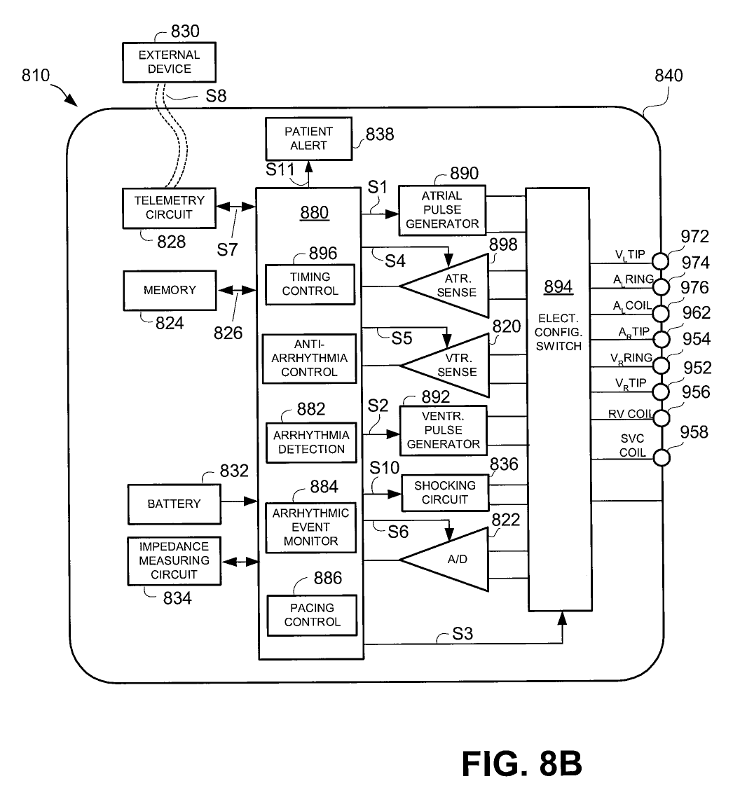

The disclosed systems and methods, which are for use in identifying and responding to potentially noisy measurements that may or may not include detection of arrhythmic episodes, are generally intended for use with subcutaneously implantable cardiac monitoring devices capable of detecting heart rate without leads invasively positioned within the heart, and with a non-implanted system that can communicate with such a cardiac monitoring device (e.g., to upload information from the cardiac monitoring device and/or reprogram the cardiac monitoring device). An exemplary subcutaneously implantable cardiac monitoring device will thus be described in conjunction with FIGS. 1A-7. However, systems and methods will further be disclosed for identifying and responding to potentially noisy measurements that may or may not include detection of arrhythmic episodes obtained with an implantable cardiac stimulation device, which is generally less affected by environmental and physiological noise than subcutaneously implanted devices, to monitor the integrity of leads positioned within the heart. An exemplary implantable cardiac stimulation device will thus be described in conjunction with FIGS. 8 and 9. It is recognized that numerous variations of cardiac monitoring devices exist, and the embodiments of the present invention are not limited to use with the exemplary devices described. For example, embodiments of the present invention can also be used with ambulatory electrocardiograph devices, such as Holter monitors, that use surface electrodes to obtain ECGs.

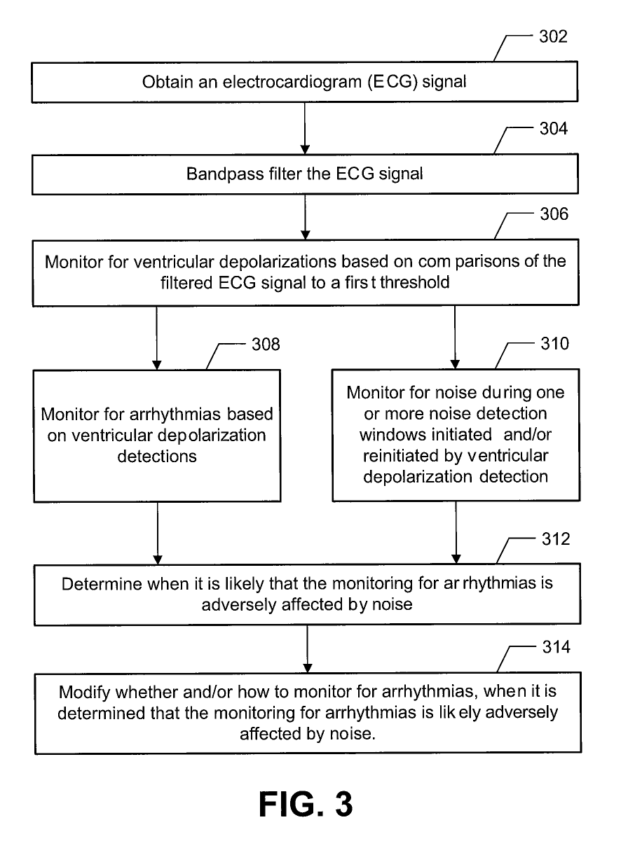

FIGS. 1A and 1B will now be used to describe subcutaneously implantable cardiac monitoring devices that can be used to identify and respond to noise during monitoring for arrhythmic events, in accordance with embodiments of the present invention. As will be described in more detail below with reference to the flow diagrams of FIGS. 3-5, the devices of FIGS. 1A-1D, can be used to obtain a subcutaneous electrocardiogram (subQ ECG) signal indicative of electrical activity of the patient's heart, and based thereon, can monitor for arrhythmic events while identifying noise.

Referring to FIGS. 1A and 1B, subcutaneously implantable cardiac monitoring devices 110 are shown as including subQ electrodes 112, which can be used to obtain subq ECG signals. Such devices are described, for example, in U.S. Ser. No. 11/848,586, entitled “Implantable Systemic Blood Pressure Measurement Systems and Methods,” (Fayram et al.), which was filed Aug. 31, 2007, and which is incorporated herein by reference. The cardiac monitoring devices 110 can optionally include an optical sensor (not shown) that can be used to produce a PPG signal used for measuring blood volume and/or blood pressure. In accordance with an embodiment, the cardiac monitoring devices 110 include suture holes 114 for fixation of the cardiac monitoring device 110 to patient tissue, which is useful for obtaining a high quality signal by reducing motion artifacts and, where an optical sensor is used, useful for applying a consistent contact force at the site of the optics to improve optical coupling with tissue. The suture holes 114 can be openings through the housing 140 (e.g., as shown in FIG. 1A), or the suture holes can be openings through tabs 116 that extend from the housing 140 (e.g., as shown in FIG. 1B). Other locations of the holes 114 and/or the tabs 116 are possible, and within the scope of the present invention. Although not shown in FIG. 1D, similar suture holes 114 can be included in the cardiac monitoring device 110 shown therein.

In the embodiments of FIGS. 1A and 1B, the subq electrodes 112 (also referred to as ECG electrodes) are located on the housing 140, and more specifically, the subq electrodes are substantially flush with and/or adjacent to the housing 140. The housing 140 can be made of a metal or other conductive material, in which case, the electrodes 112 should be electrically isolated from the housing 140. Alternatively, the housing 140 can be made from a non-conductive material, e.g., a plastic or other polymer. In certain embodiments, the cardiac monitoring devices 110 are not capable of pacing and not capable of defibrillation, but rather, the implantable devices 110 are primarily for monitoring purposes.

In FIG. 1A, the oblong geometry of the housing 140a can be used to maximize separation of electrodes 112 and prevent rotation of the device within a tissue pocket, thereby allowing interpretation of morphology features in an ECG sensed using the electrodes 112. While two subQ electrodes 112 are shown, more can be present. The housing 140 is preferably small and thin, with smooth surfaces and a physiologic contour that minimizes tissue trauma and inflammation. If desired, though not necessary, sutures or some other fixation means can be used to fix the housing 140 at a specific implanted location.

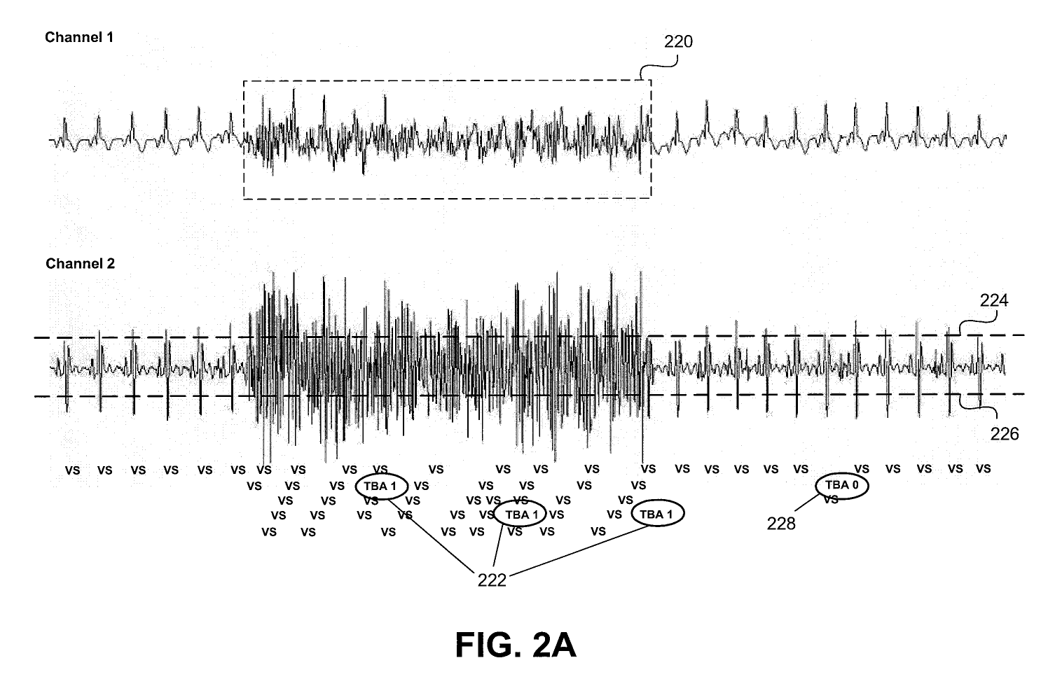

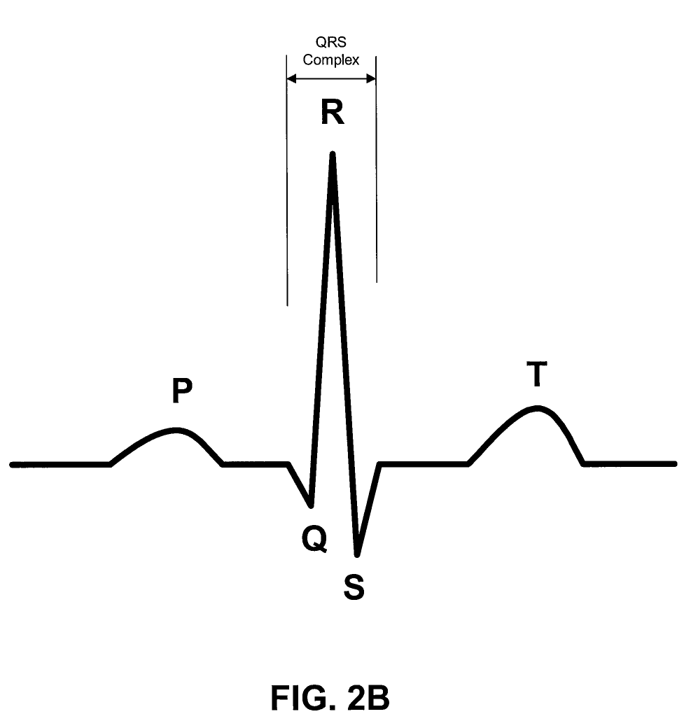

FIG. 1B illustrates an embodiment where the housing 140b is a different shape, and which includes an additional subQ electrode 112. In the embodiment illustrated in FIG. 1B, three subQ electrodes 112 are present, one at each apex of the triangle formed by the device housing 140. If the device is implanted with an appropriate orientation, these three electrodes may allow the three standard surface ECG leads to be approximated. In another embodiment, four or more ECG electrodes 112 might be used. U.S. Pat. No. 6,409,675 (Turcott), which is incorporated herein by reference, in its discussion of FIGS. 2a-2c and 3a-3c provides some additional details of a subcutaneously implantable cardiac monitoring device that includes ECG electrodes on its housing.

FIG. 1C illustrates the subcutaneously implantable cardiac monitoring device 110b of FIG. 1B, implanted in a subcutaneous pectoral region of a patient 108. An alternative location where the device 110b can be implanted includes, but is not limited to, a subcutaneous abdominal region.

FIG. 1D illustrates an alternative embodiment wherein the subQ electrodes 112 are remote from the housing, yet are still extracardiac. While the device 110d shown in FIG. 1D may be more complicated to implant than the devices 110a and 110b shown in FIGS. 1A and 1B, because the device 110d and electrodes 112 in FIG. 1D are still extracardiac and subcutaneous, the device 110d and remote subQ electrodes 112 can still be more easily and quickly implanted than an ICD, for example. In the embodiment of FIG. 1D, the subq extracardiac electrodes 112 are preferably extravascular and can be, e.g., paddle electrodes mounted subcutaneously outside of the rib cage, but are not limited thereto. Exemplary locations of the subQ extracardiac electrodes 112 include near the bottom of the sternum (slightly to the left), below the left pectoral area, and below the clavicle and on the back left side (just below the shoulder blade). Additional and/or alternative locations for subQ electrodes 112 are also within the scope of the present invention. Also, while one or more electrodes 112 can be remote from the housing 140, as shown in FIG. 1D, one or more electrodes can be on the housing 140 (e.g., substantially flush with and/or adjacent to the housing 140), as was discussed above with reference to FIGS. 1A and 1B, and/or a conductive housing 140 can act as one of the electrodes.

As noted above, cardiac monitoring devices implanted subcutaneously can be susceptible to baseline wander, environmental noise, and physiological noise. Severe noise levels may result in inappropriate ventricular depolarization detection, which can lead, e.g., to false VT and/or AF detections. Several measures can be taken in the design of the subcutaneously implantable cardiac monitoring device to reduce the effects of noise. For example, the detection electrodes can be arranged on opposite sides of the cardiac monitoring device, so that, in the implant position between the muscle layer and the fat layer, one electrode will face the muscle layer, while another electrode will face the fat tissue layer. This helps avoid direct differential measurement from the muscle, which might be especially susceptible to muscle activity-induced noise. However, the position of the electrodes does not completely eliminate noise.

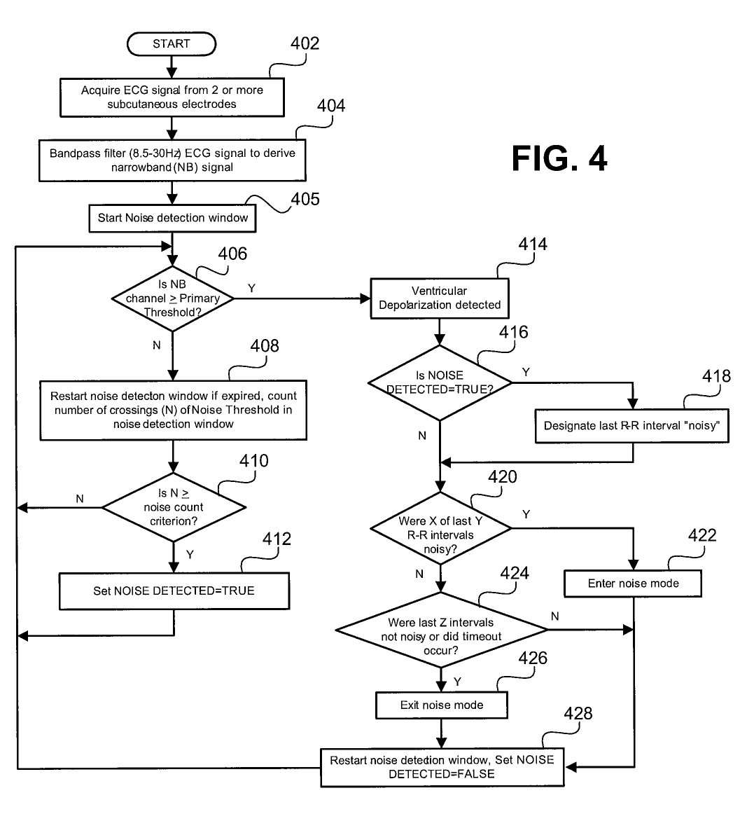

FIG. 2A is an ECG of a person obtained using a subcutaneously implantable cardiac monitoring device resembling the cardiac monitoring device 110a as described above with reference to FIG. 1A. Channel 1 illustrates a wideband ECG signal obtained using the electrodes of the cardiac monitoring device. The amplifier circuits of the cardiac monitoring device that sensed the ECG signal used bandpass filtering to achieve a passband of 0.5 Hz to 30 Hz. FIG. 2B is an example of one cycle of a typical ECG signal. Ventricular depolarization can be detected, e.g., by detecting the Q wave of the QRS complex, the R wave of the QRS complex, and/or the S wave of the QRS complex. However, since the R wave is the easiest to detect, due to its relatively large magnitude, it is practical for ventricular depolarization to be detected by detecting the R wave. Accordingly, any known or future developed technique for detecting an R wave (e.g., by peak detection or threshold crossing) can be used to detect ventricular depolarization. Exemplary techniques for detecting R waves are disclosed in the discussion of FIGS. 4-8 in U.S. Pat. No. 7,403,813, entitled “Systems and Methods for Detection of VT and VF from Remote Sensing Electrodes” (Farazi et al.), which patent is incorporated herein by reference. Alternatively, known or future developed techniques for detecting the Q, R and/or S waves can be used to detect ventricular depolarization.

Referring again to FIG. 2A, channel 2 illustrates a narrowband ECG signal generated by further bandpass filtering the signal obtained using the electrodes of the cardiac monitoring device to restrict the response to the 8.5 Hz to 30 Hz range. Further bandpass filtering is intended to pass the QRS complexes of the ECG signal and reject other components, such as the P-wave and T-wave components of the signal. The narrowband ECG signal attenuates noise sources such as baseline wander and respiration and most muscle activity-induced noise (a majority of the energy of muscle activity-induced noise is in the 20 Hz to 80 Hz range), as well as electrical power line noise at 50 Hz/60 Hz.

Even after signal conditioning, in some patients an obtained ECG signal may still be subject to severe environmental noise or experience severe muscle activity-induced noise which may present noise interference to ventricular depolarization detection. The ECG signal shown in of FIG. 2A extends over a period of time of about 24 seconds. A portion 220 of the ECG signal of FIG. 2A shows muscle activity-induced noise generated by muscle activity that cannot be detected by a separate activity sensor (such as an accelerometer or other one-dimensional motion sensor). The ECG signal during this period is influenced by isometric activity of the person. Specifically, the person's hands were pressed against each another, creating intense muscle activity in the arms and chest, which caused noise in the ECG. It should be noted that embodiments of systems and methods in accordance with the present invention can further comprise an activity sensor (e.g. a one-dimensional motion sensor, or multi-dimensional sensor) to enable robust detection of noise sources using multiple techniques.

As can be seen in Channel 2 of FIG. 2A, an arrhythmic event monitor of the cardiac monitoring device (or of an external device to which the ECG data is uploaded) identifies each ventricular depolarization by an R wave in an ECG signal, marking the R wave with “VS” to denote a ventricular sensed signal. The R wave can be identified when the ECG signal exceeds a first and/or a second threshold (for example the upper threshold 224 and/or the lower threshold 226 overlaying the ECG trace of Channel 2). In an embodiment where the ECG signal is rectified, a single threshold is applied to identify the R wave. The arrhythmic event monitor detects what the monitor interprets as a tachycardia event within the portion 220 known to be affected by noise when the R wave frequency exceeds a pre-defined limit. The arrhythmic event monitor marks the detected tachycardia event “TBA 1” to denote tachyarrhythmias, bradyarrhythmias or asystole events at three locations 228 along the ECG trace until the arrhythmic event monitor determines that the tachycardia event is no longer detected. The arrhythmic event monitor then marks the end of the event “TBA 0” at a location 228 along the ECG after the portion 220.

Frequent false detection of arrhythmias can be problematic for multiple reasons. ECG traces can be stored in the memory of the cardiac monitoring device for retrieval by a physician at a later date. However, the cardiac monitoring device preferably should be made as small as is practical. As such, memory should be limited in size so that there is finite storage onboard the cardiac monitoring device. ECG traces (along with metadata associated with the ECG traces identifying, among other things, date and time of day) are therefore recorded and stored following detection of an arrhythmic event. Frequent false detection can cause storage to fill up much faster with ECG traces, causing true episodes stored earlier to be erased (where the recording function operates on a loop). Further, frequent false detection also wastes a physician's time by prompting the physician to confirm whether a detected event is a true arrhythmic event.

FIG. 3 is a flowchart of an embodiment of a method in accordance with the present invention for detecting and responding to noise events in an ECG. The method is especially useful to detect and respond to noise events associated with myopotential having a frequency typically ≧20 Hz. Further, the method can be useful to detect and respond to noise events associated with other sources, such as environmental noise. A subcutaneously implanted cardiac monitoring device obtains an ECG signal (Step 302). The ECG signal is bandpass filtered to remove noise attributable to many noise sources and to remove lower frequency components from the ECG. For example, bandpass filtering can remove at least the P-wave and T-wave components of the ECG (Step 304). The cardiac monitoring device monitors for ventricular depolarization based on comparisons of the filtered ECG signal to a first threshold (Step 306) and arrhythmias based on the frequency of ventricular depolarization detections (Step 308). The first threshold can be set to reliably detect a ventricular depolarization having a profile ordinary to the patient. In a specific embodiment, a ventricular depolarization can be detected if the first threshold is exceeded in the positive or negative direction.

Following each detected ventricular depolarization, the cardiac monitoring device monitors for noise during one or more noise detection windows (Step 310). Monitoring for noise can occur within one long window or a plurality of back-to-back windows. In an embodiment, if a noise window is not completed when a ventricular depolarization is detected, the noise window is interrupted and the one or more noise detection windows are reset. It is not physiologically possible in a normal human being for two ventricular depolarizations to occur within a window as narrow as 125 ms. If two or more ventricular depolarization events are detected occurring within 125 ms of one another, one or more of the events is a false detection attributable to noise. Thus, in an embodiment the ECG can be monitored in continuous recurring windows spanning a length of time set between 90 ms and 250 ms (preferably 125 ms). Alternatively, the one or more windows can be longer or shorter, depending on the patient and the condition the physician is seeking to diagnose. In an embodiment, a noise count is incremented in response to the ECG signal amplitude crossing a second threshold. In an embodiment, the second threshold can be set to a fraction of the most sensitive sense detection threshold (i.e. maximum sensitivity setting). For example, the second threshold can be set between 25% and 85% (preferably 75%) of the maximum sensitivity setting. The maximum sensitivity setting is a programmable setting that defines the lowest amplitude R wave detectable by the cardiac monitoring device, although preferably the maximum sensitivity setting is set above the lowest amplitude signal the cardiac monitoring device is physically capable of measuring. In an embodiment, the maximum sensitivity setting and the first threshold can be synonymous. In some embodiments, the second threshold can be a moving value that can be varied by the cardiac monitoring device based on analysis of a pre-defined number of recent ECG traces. For example, techniques for dynamically adjusting a threshold based on R wave amplitude are described in U.S. Pat. No. 7,403,813, entitled “Systems and Methods for Detection of VT and VF from Remote Sensing Electrodes” (Farazi et al.), which was incorporated herein by reference above. Alternatively, the second threshold can be set (and reset) by the physician.

If the noise count within the one or more noise detection windows exceeds a prescribed limit, the R-to-R interval including the one or more noise detection windows can be designated noisy. In an embodiment, the prescribed limit can be a cumulative number across one or more noise detection windows. In a specific embodiment, back-to-back 125 ms windows are used and the prescribed limit can be four noise counts (programmable) or more (corresponding to a noise frequency of 20 Hz or higher). In an alternative embodiment, the prescribed limit can be based on noise count trends, for example the prescribed limit can be based on a number of noise detections occurring for M out of the last N immediately preceding noise detection windows.

The cardiac monitoring device determines that monitoring for arrhythmias is likely to be adversely affected by noise (Step 312) when a criterion is satisfied to indicate that the ECG signal is noisy, and in response the cardiac monitoring device can modify whether and/or how to monitor for arrhythmias within the ECG signal (Step 314). In some embodiments, the cardiac monitoring device can disallow storage of ECG traces and associated data while the ECG signal is flagged as likely affected by noise. In other embodiments, the ECG traces can be recorded by the cardiac monitoring device, but the ECG trace can be marked with annotations indicating detection of noise events. When a criterion is satisfied to indicate that the ECG signal is no longer affected by noise, the cardiac monitoring device can allow storage of ECG traces. The criterion for modifying monitoring for arrhythmias and criterion for resuming normal monitoring for arrhythmias can be different or the complementary. In an embodiment, the criterion satisfying a determination that the ECG signal is not affected by noise can include determining—that X out of the last Y immediately preceding R-to-R intervals are not noisy, where Y is an integer ≧2, and X is an integer ≦Y. For example, in a specific embodiment, when the cardiac monitoring device determines that the four out of five immediately preceding R-to-R intervals are free from noise, the criterion to resume normal monitoring for arrhythmias is satisfied. In the specific embodiment, monitoring for arrhythmias can be modified under a complementary criterion, i.e. if the cardiac monitoring device determines that the four out of five immediately preceding R-to-R intervals are noisy. Alternatively, the conditions to modify arrhythmia monitoring can be different, e.g. the conditions can comprise determining that three consecutive R-to-R intervals are noisy. In an alternative embodiment, the criterion to modify monitoring for arrhythmias and/or to resume normal monitoring for arrhythmias can include, for example, a minimum number of consecutive noise detection windows (rather than consecutive R-to-R intervals) with a noise count above or below the prescribed limit.

FIG. 4 is a detailed flowchart of a specific embodiment of a method in accordance with the present invention for detecting and responding to noise events in an ECG including logical steps that can be encoded as an instruction set on a machine readable storage medium for execution by a microprocessor of the cardiac monitoring device. The cardiac monitoring device obtains an ECG signal from two or more subcutaneously implanted electrodes (Step 402). As described above with reference to FIGS. 1A-1D, the electrodes can be positioned on a housing of the cardiac monitoring device, can be remote from the housing of the cardiac monitoring device, or alternatively one or more electrodes can be positioned on the housing and one or more electrodes can be remote from the housing. It is also possible that the electrodes are surface electrodes. The ECG signal is bandpass filtered to remove noise attributable to many noise sources and to remove lower frequency components from the ECG. For example, bandpass filtering can remove at least the P-wave and T-wave components of the ECG (Step 404). The cardiac monitoring device continuously monitors for noise in the ECG signal. Noise monitoring is initiated when the ECG is acquired and/or filtered and includes starting a noise detection window (Step 405) within which noise threshold crossings can be monitored. Concurrently, the cardiac monitoring device monitors for a ventricular depolarization based on comparisons of the narrowband ECG signal to a primary threshold (Step 406). If the narrowband ECG signal meets or exceeds the primary threshold, a ventricular depolarization is detected (Step 414). If the narrowband ECG signal is below the primary threshold, the number of threshold crossings of a noise threshold is counted within a noise detection window (Step 408). If the noise detection window is expired, the noise detection window is restarted, so that the noise window is recurring. The noise threshold is preferably a fraction of the maximum sensitivity setting. For example, as above, the noise threshold can be set between 25% and 85% (preferably 75%) of the maximum sensitivity setting. As above, in some embodiments, the noise threshold can be a moving value that can be varied by the cardiac monitoring device based on analysis of ECG traces recent by a pre-defined measure of time to the analysis. Also, if the primary threshold varies, the noise threshold can vary as a function of the varying primary threshold (e.g., by having the noise threshold be a percentage, such as 75%, of the varying primary threshold). Alternatively, the noise threshold can be set (and reset) by the physician.

The cardiac monitoring device monitors for noise based on comparisons of the number of noise threshold crossings (the noise count, “N”) with a noise count criterion (Step 410). If the noise count meets or exceeds the noise count criterion, the variable NOISE DETECTED is set to TRUE (Step 412). As above, in an embodiment, the noise count criterion can be based on a noise count cumulative across one or more noise detection windows or a noise count trend comprising noise detections occurring for M out of the last N immediately preceding noise detection windows. The cardiac monitoring device then resumes monitoring the narrowband ECG signal for primary threshold crossings (Step 406). If the noise count is below the noise count criterion, the variable NOISE DETECTED remains at its previous setting, and the cardiac monitoring device resumes monitoring the narrowband ECG signal for primary threshold crossings (Step 406).

If the variable NOISE DETECTED is set to TRUE and a ventricular depolarization is detected, the last R-R interval is designated as “noisy” (Step 416). If the variable NOISE DETECTED is set to FALSE, the last R-R interval is not designated as “noisy.” The cardiac monitoring device then determines if X of the last Y (e.g., 3 of the last 5) immediately preceding R-R intervals are noisy (Step 420). If the condition checked at step 420 is satisfied, the cardiac monitoring device enters noise mode (or remains in noise mode) (Step 422), the noise detection window is restarted, and variable NOISE DETECTED is set to FALSE (Step 428). The cardiac monitoring device then resumes monitoring the narrowband ECG signal for primary threshold crossings (Step 406).

If the condition checked at step 420 is not satisfied, the cardiac monitoring device determines if the last Z R-R intervals (e.g., Z=2) are noisy or if a timeout has occurred (Step 424). If the last Z R-R intervals are not noisy or if a timeout has occurred, the cardiac monitoring device exits noise mode (Step 426). If the last Z (e.g., Z=2) R-R intervals are noisy and a timeout did not occur, the cardiac monitoring device remains in its current mode, the noise detection window is restarted, and variable NOISE DETECTED is set to FALSE (Step 428). The cardiac monitoring device then resumes monitoring the narrowband ECG signal for primary threshold crossings (Step 406).

In noise mode, the cardiac monitoring device can monitor signals with multiple different modifications to the cardiac monitoring device's response. For example, the cardiac monitoring device can enter a “monitor” noise mode, marking the recorded and/or displayed ECG with a noise entry mark. Alternatively, the cardiac monitoring device can enter an “inhibit” noise mode, and inhibit the recording and storage of ECG traces until the cardiac monitoring device exits noise mode. Both marking the ECG and inhibiting recording and storage of ECG traces can provide the benefit of improving accuracy of diagnosis and can reduce the time required of a physician to analyze the ECG traces and match the ECG traces to patient activity. Inhibiting recording and storage of ECG traces can further provide the benefit of reducing an amount of storage space required for noisy ECG traces which can be rendered unusable by such noise.

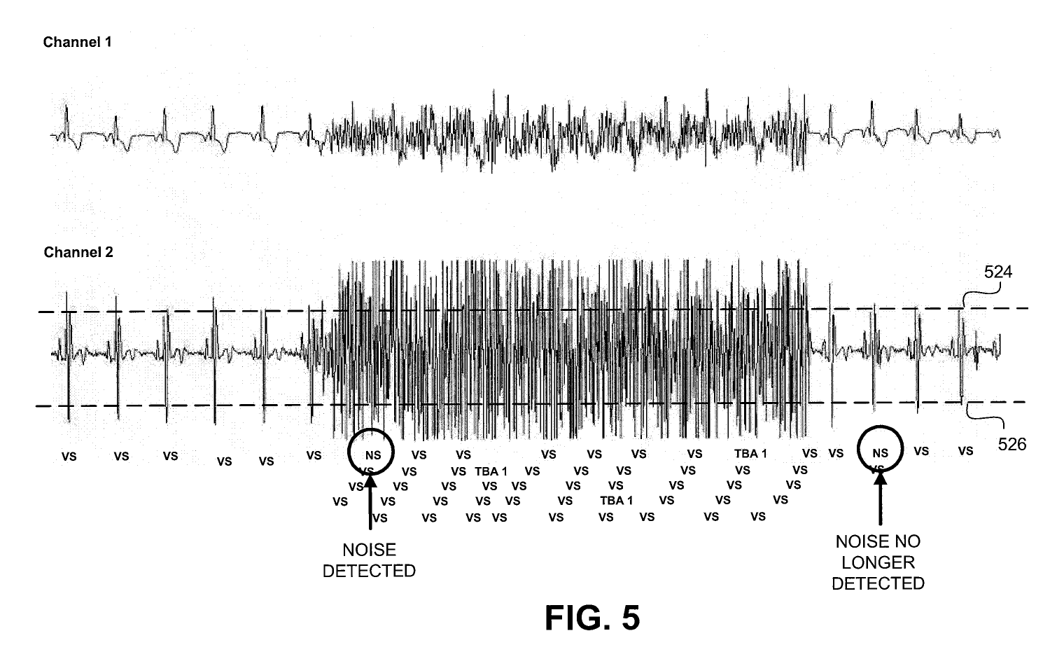

FIG. 5 is an ECG signal that is processed in accordance with an embodiment of the present invention. The ECG signal is obtained using a subcutaneously implantable cardiac monitoring device resembling cardiac monitoring devices as described above in FIGS. 1A-1C. Channel 1 illustrates a wideband ECG obtained using the electrodes of the cardiac monitoring device having a passband of 0.5 Hz to 30 Hz. Channel 2 illustrates a narrowband ECG generated by bandpass filtering the signal obtained using the electrodes of the cardiac monitoring device to restrict the response to the 8.5 Hz to 30 Hz range. The ECG shown in of FIG. 5 extends over a period of time of about 40 seconds. As above, a portion of the ECG of FIG. 5 shows muscle activity-induced noise generated by muscle activity that cannot be detected by a separate activity sensor. The ECG during this period is influenced by isometric activity of the person. Specifically, the person's hands were pressed against each another, creating intense muscle activity in the arms and chest, which created noise in the ECG.

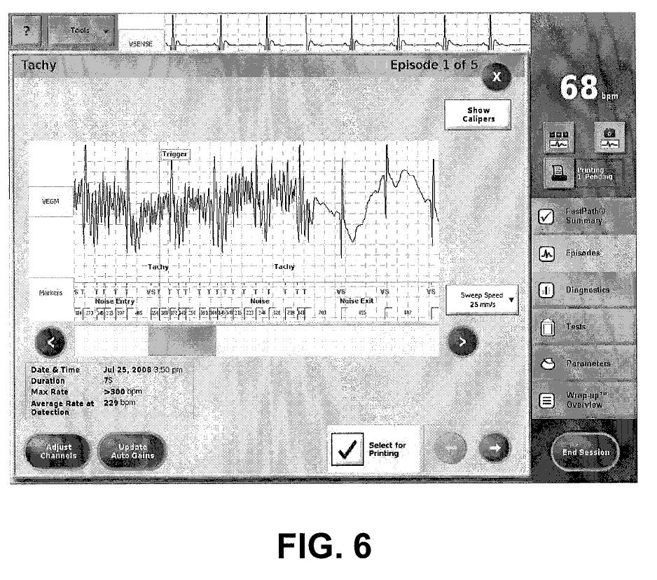

As can be seen in Channel 2 of FIG. 5, an arrhythmic event monitor of the cardiac monitoring device identifies each crossing of the threshold 524 and/or the threshold 526 as a ventricular depolarization detected in the ECG, marking each with “VS.” The arrhythmic event monitor further detects what the monitor interprets as a tachycardia event within the portion of the ECG known to be affected by noise. The arrhythmic event monitor marks the detected tachycardia event “TRA T” along the ECG until the monitor determines that the tachycardia event is no longer detected. However, the arrhythmic event monitor also detects the presence of noise based on the method described with reference to FIG. 4 and enters noise mode. Entering noise mode, the arrhythmic event monitor marks the ECG trace “NS” to indicate the beginning of a period of noise. When the noise event has passed and the arrhythmic event monitor no longer detects noise as determined by applying the method of FIG. 4, the monitor marks the ECG trace “NS” to indicate that the arrhythmic event monitor has exited noise mode. Alternative noise entry and noise exit markers can also be used In an embodiment the Noise Entry marker and the Noise Exit marker can be different from one another so that they are easily distinguishable, e.g., as shown in FIG. 6 discussed below.

FIG. 6 is a screenshot from a programmer usable to program, display, and/or analyze ECG traces for an embodiment of a cardiac monitoring device and method of monitoring arrhythmic events in accordance with the present invention. The screenshot displays over-detection of ventricular depolarizations leading to false detections of ‘tachycardia’ episodes during isometric exercise. The arrhythmic event monitor enters noise mode, labeled “Noise Entry,” when threshold crossings exceed a predefined frequency (coincident with the isometric exercise), and subsequently exits noise mode, labeled “Noise Exit,” when conditions are satisfied.

### Exemplary Subcutaneously Implantable Cardiac Monitoring Device

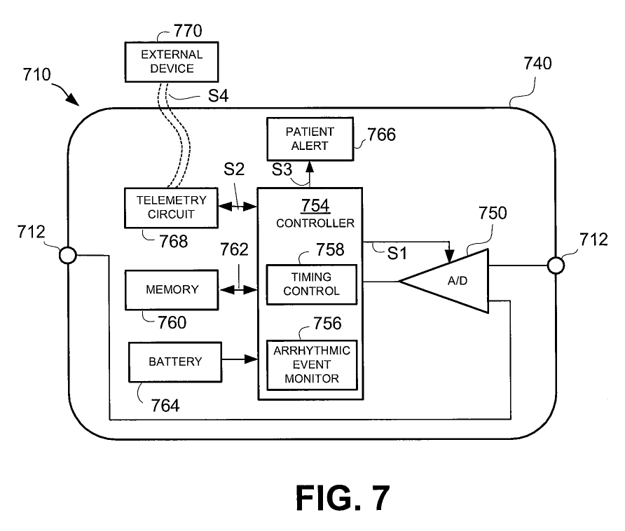

FIG. 7 is a simplified block diagram of an embodiment of a subcutaneously implantable cardiac monitoring device 710 for use with systems and methods in accordance with the present invention. While a particular cardiac monitoring device is shown, this is for illustration purposes only, and one of ordinary skill in the art could readily add, duplicate, eliminate or disable the circuitry in any desired combination.

The housing 740 for the subcutaneously implantable cardiac monitoring device 710, shown schematically, is often referred to as the “can” or “case.” The cardiac monitoring device 710 detects electrical signals from a patient's heart by way of two subq electrodes 712.

At the core of the cardiac monitoring device 710 is a programmable microcontroller 754 which controls ECG signal detection, ECG signal monitoring, ECG trace storage, and other cardiac monitoring device controls. As is well known in the art, the microcontroller 754 typically includes a microprocessor, or equivalent control circuitry, designed specifically for controlling signal detection and signal monitoring and can further include RAM or ROM memory 760, logic and timing control circuitry 758, state machine circuitry, and I/O circuitry. Typically, the microcontroller 754 includes the ability to process or monitor input signals (data) as controlled by a program code stored in a designated block of memory. The details of the design of the microcontroller 754 are not critical to the present invention. Rather, any suitable microcontroller 754 can be used to carry out the functions described herein. The use of microprocessor-based control circuits for data analysis functions are well known in the art. In specific embodiments of the present invention, the microcontroller 754 performs some or all of the steps associated with arrhythmia detection. Further, timing control circuitry 758 which is used to keep track of noise detection windows, alert intervals, marker channel timing, etc., is well known in the art.

Cardiac signals are applied to the inputs of an analog-to-digital (A/D) data acquisition system 750 electrically connected with the electrodes 712. The data acquisition system 750 is configured to acquire ECG signals, convert the raw analog data into a digital signal, and store the digital signals or portions thereof for later processing and/or telemetric transmission to an external device 770.

The data acquisition system 750 preferably employs one or more low power, precision amplifiers with programmable gain and/or automatic gain control, bandpass filtering, and a threshold detection circuit, as known in the art, to selectively sense the cardiac signal of interest. The automatic gain control enables the device 710 to deal effectively with the difficult problem of sensing low amplitude signals. Alternatively, an automatic sensitivity control circuit may be used to effectively deal with signals of varying amplitude. The data acquisition system 750 can be used to perform the bandpass filtering used in embodiments of the present invention. Alternatively, one or more other filters can be used.

The outputs of the data acquisition system 750 are connected to the microcontroller 754 which, in turn, is able to analyze cardiac signals. The data acquisition system 750, in turn, receives control signals S1, from the microcontroller 754 for purposes of controlling the gain, threshold, polarization charge removal circuitry (not shown), and timing of any blocking circuitry (not shown) coupled to the inputs of the data acquisition system 750.

For arrhythmia detection, the cardiac monitoring device 710 includes an arrhythmic event monitor 756, which can analyze the timing intervals between sensed events (e.g., ventricular depolarization) to compare them to predefined rate zone limits (e.g., bradycardia, normal, tachycardia), and further to determine, as described above with reference to FIGS. 3 and 4, whether the ECG signal is noisy and should enter or exit a noise mode.

The arrhythmic event monitor 756 can be implemented within the microcontroller 754, as shown in FIG. 7. Thus, the arrhythmic event monitor 756 can be implemented by software, firmware, or combinations thereof. It is also possible that all, or portions, of the arrhythmic event monitor 756 can be implemented using hardware. Further, it is also possible that all, or portions, of the arrhythmic event monitor 756 can be implemented separate from the microcontroller 754.

In accordance with embodiments of the present invention, the subcutaneously implantable cardiac monitoring device 710 can store, in memory 760, ECG traces along with information regarding whether noise and/or arrhythmic episodes were detected based on the ECG traces. Additional details of an exemplary implantable system including extracardiac subcutaneous electrodes are described in U.S. Pat. No. 7,403,813, entitled “Systems and Methods for Detection of VT and VF from Remote Sensing Electrodes” (Farazi et al.), which was incorporated herein by reference above.

The microcontroller 754 is further coupled to the memory 760 by a suitable data/address bus 762, wherein the programmable operating parameters used by the microcontroller 754 are stored and modified, as required, in order to customize the operation of the implantable device 710 to suit the needs of a particular patient. Such operating parameters define, for example, arrhythmia detection criteria and noise response mode.

The operating parameters of the subcutaneously implantable cardiac monitoring device 710 may be non-invasively programmed into the memory 760 through a telemetry circuit 768 in telemetric communication via communication link S4 with an external device 770, such as a programmer, transtelephonic transceiver, and/or a diagnostic system analyzer. The telemetry circuit 768 can be activated by the microcontroller 754 by a control signal S2. The telemetry circuit 768 advantageously allows ECG signal data, and status information relating to the operation of the device 710 (as contained in the microcontroller 754 or memory 760) to be sent to the external device 770 through an established communication link S4. In specific embodiments of the present invention, ECG signal data which correspond to detected potential episodes of an arrhythmia (e.g., tachycardia), are sent to the external device 770 through the established communication link S4.

For examples of telemetry devices, see U.S. Pat. No. 4,809,697, entitled “Interactive Programming and Diagnostic System for use with Implantable Pacemaker” (Causey, III et al.); U.S. Pat. No. 4,944,299, entitled “High Speed Digital Telemetry System for Implantable Device” (Silvian); and U.S. Pat. No. 6,275,734 entitled “Efficient Generation of Sensing Signals in an Implantable Medical Device such as a Pacemaker or ICD” (McClure et al.), which patents are hereby incorporated herein by reference.

The implantable device 710 additionally includes a battery 764 which provides operating power to all of the circuits shown in FIG. 7. The battery 764 should also have a predictable discharge characteristic so that elective replacement time can be detected. The implantable device 710 can also include a magnet detection circuitry (not shown), coupled to the microcontroller 754. It is the purpose of the magnet detection circuitry to detect when a magnet is placed over the implantable device 710, which magnet may be used by a clinician to perform various test functions of the implantable device 710 and/or to signal the microcontroller 754 that the external programmer 770 is in place to receive or transmit data to the microcontroller 754 through the telemetry circuit 768.

The above described implantable device 710 was described as an exemplary device. One or ordinary skill in the art would understand that embodiments of the present invention can be used with alternative types of implantable devices. Accordingly, embodiments of the present invention should not be limited to use only with the above described devices.

### Exemplary Implantable Cardiac Stimulation Device

FIG. 8A will now be used to describe an exemplary implantable cardiac stimulation device 810 that can be used to identify and respond to noise potentially associated with defects in the device or leads connected to the device during monitoring for arrhythmic events, in accordance with embodiments of the present invention. The implantable cardiac stimulation device 810 is shown comprising an implantable stimulation device, which can be a pacing device and/or an ICD. The implantable cardiac stimulation device 810 is shown as being in electrical communication with a patient's heart by way of three leads 850, 860, and 870, which can be suitable for delivering multi-chamber stimulation and shock therapy.

To sense atrial cardiac signals and to provide right atrial chamber stimulation therapy, the implantable cardiac stimulation device 810 is coupled to an implantable right atrial lead 860 having at least an atrial tip electrode 862, which typically is implanted in the patient's right atrial appendage. To sense left atrial and ventricular cardiac signals and to provide left-chamber pacing therapy, the implantable cardiac stimulation device 810 is coupled to a “coronary sinus” lead 870 designed for placement in the “coronary sinus region” via the coronary sinus for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, an exemplary coronary sinus lead 870 is designed to receive atrial and ventricular cardiac signals and to deliver left ventricular pacing therapy using at least a left ventricular tip electrode 872, left atrial pacing therapy using at least a left atrial ring electrode 874, and shocking therapy using at least a left atrial coil electrode 876.

The implantable cardiac stimulation device 810 is also shown in electrical communication with the patient's heart by way of an implantable right ventricular lead 850 having, in this embodiment, a right ventricular tip electrode 852, a right ventricular ring electrode 854, a right ventricular (RV) coil electrode 856, and an SVC coil electrode 858. Typically, the right ventricular lead 850 is transvenously inserted into the heart so as to place the right ventricular tip electrode 852 in the right ventricular apex so that the RV coil electrode 856 will be positioned in the right ventricle and the SVC coil electrode 858 will be positioned in the superior vena cava. Accordingly, the right ventricular lead 850 is capable of receiving cardiac signals and delivering stimulation in the form of pacing and shock therapy to the right ventricle.

Inappropriate therapy, particularly inappropriate shock therapy, applied in response to false VT/VF detection, for example, can negatively impact a patient's quality of life, induce true ventricular arrhythmia (e.g. by shocking on a T-wave portion of a cycle), and/or unnecessarily reduce longevity of the cardiac stimulation device by expending battery power. False VT/VF detection can be caused by environmental and/or physiological noise, as described above, and/or noise caused by defects of the implantable cardiac stimulation device 810, specifically lead fractures.

Referring back to FIG. 2A, channel 2 illustrates a narrowband ECG signal generated by bandpass filtering the signal obtained using the electrodes of the cardiac monitoring device to restrict the response to the 8.5 Hz to 30 Hz range. The narrowband ECG signal attenuates noise sources such as baseline wander and respiration and most muscle noise (a majority of the energy of muscle noise is in the 20 Hz to 80 Hz range), as well as electrical power line noise at 50 Hz/60 Hz. Similarly, a narrowband intracardiac electrogram (IEGM) signal attenuates noise sources such as baseline wander and respiration. However, a lead fracture can generate noise some part of which is in the 15-20 Hz range, which can be falsely detected as a VF. Inappropriate shock therapy may be delivered in response to the falsely detected VF. A more practical noise detection algorithm is needed for effectively reducing false VT/VF sensing to avoid inappropriate shock therapy.

Embodiments of methods in accordance with the present invention for detecting and responding to noise events described above with reference to FIGS. 3 and 4 can be applied to detect and respond to noise associated with lead fracture(s) affecting an IEGM signal. In noise mode, the implantable cardiac stimulation device can monitor IEGM signals for noise with multiple different modifications to the implantable cardiac stimulation device's response. For example, the implantable cardiac stimulation device can enter an “inhibit” noise mode which inhibits the application of shock and/or other stimulation (e.g., anti-tachycardia pacing) therapy during noise mode. A number of entries of noise mode can be recorded by the implantable cardiac stimulation device, and the data, when uploaded to an external device (e.g., 770 in FIG. 7 or 830 in FIG. 8B), can prompt a physician to evaluate the implantable cardiac stimulation device and leads for defects.

When conditions, such as described above, are met, the implantable cardiac stimulation device can exit noise mode and resume therapeutic response. In an embodiment, conditions for exiting noise mode can comprise two consecutive cardiac cycles with no noise detection are found, or an elapsed time of 1.2 seconds (programmable) since the last noisy cardiac cycle. One of ordinary skill in the art, upon reflecting on the above will appreciate the different conditions that can be used to trigger noise mode on and/or off. Embodiments of methods in accordance with the present invention can inhibit potentially inappropriate shock and/or other stimulation therapy to improve a patient's quality of life and reduce device wear.

FIG. 8B will now be used to provide some exemplary details of the components of the implant device 810. Referring now to FIG. 8B, the above implant device 810, and alternative versions thereof, can include a microcontroller 880. As is well known in the art, the microcontroller 880 typically includes a microprocessor, or equivalent control circuitry, and can further include RAM or ROM memory, logic and timing circuitry, state machine circuitry, and I/O circuitry. Typically, the microcontroller 880 includes the ability to process or monitor input signals (data) as controlled by a program code stored in a designated block of memory. The details of the design of the microcontroller 880 are not critical to the present invention. Rather, any suitable microcontroller 880 can be used to carry out the functions described herein. The use of microprocessor-based control circuits for performing timing and data analysis functions are well known in the art. In specific embodiments of the present invention, the microcontroller 880 performs some or all of the steps associated with monitoring blood perfusion to an organ of interest within a patient, monitoring blood volume and tumor growth in an organ, and/or monitoring for sepsis.

Representative types of control circuitry that may be used with the invention include the microprocessor-based control system of U.S. Pat. No. 4,940,052 (Mann et. al.) and the state-machines of U.S. Pat. No. 4,712,555 (Sholder) and U.S. Pat. No. 4,944,298 (Sholder). For a more detailed description of the various timing intervals used within the pacing device and their inter-relationship, see U.S. Pat. No. 4,788,980 (Mann et. al.). The '052, '555, '298 and '980 patents are incorporated herein by reference.

Depending on implementation, the implant device 810 can be capable of treating both fast and slow arrhythmias with stimulation therapy, including pacing, cardioversion and defibrillation stimulation. While a particular multi-chamber device is shown, this is for illustration purposes only, and one of skill in the art could readily duplicate, eliminate or disable the appropriate circuitry in any desired combination to provide a device capable of treating the appropriate chamber(s) with pacing, cardioversion and defibrillation stimulation. For example, where the implantable device is a monitor that does not provide any therapy, it is clear that many of the blocks shown may be eliminated.

The housing 840, shown schematically in FIG. 8B, is often referred to as the “can”, “case” or “case electrode” and may be programmably selected to act as the return electrode for all “unipolar” modes. The housing 840 may further be used as a return electrode alone or in combination with one or more of the coil electrodes, 856 and 858, 876, for shocking purposes. The housing 840 can further include a connector (not shown) having a plurality of terminals, 952, 954, 956, 958, 962, 972, 974, and 976 (shown schematically and, for convenience, the names of the electrodes to which they are connected are shown next to the terminals). As such, to achieve right atrial sensing and pacing, the connector includes at least a right atrial tip terminal (A_{R} TIP) 962 adapted for connection to the atrial tip electrode 862.

To achieve left atrial and ventricular sensing, pacing and shocking, the connector includes at least a left ventricular tip terminal (V_{L} TIP) 972, a left atrial ring terminal (A_{L} RING) 974, and a left atrial shocking terminal (A_{L} COIL) 976, which are adapted for connection to the left ventricular ring electrode 872, the left atrial tip electrode 874, and the left atrial coil electrode 876, respectively.

To support right ventricle sensing, pacing and shocking, the connector further includes a right ventricular tip terminal (V_{R} TIP) 952, a right ventricular ring terminal (V_{R} RING) 954, a right ventricular shocking terminal (R_{V} COIL) 956, and an SVC shocking terminal (SVC COIL) 958, which are adapted for connection to the right ventricular tip electrode 852, right ventricular ring electrode 854, the RV coil electrode 856, and the SVC coil electrode 858, respectively.

An atrial pulse generator 890 and a ventricular pulse generator 892 generate pacing stimulation pulses for delivery by the right atrial lead 860, the right ventricular lead 850, and/or the coronary sinus lead 870 via an electrode configuration switch 894. It is understood that in order to provide stimulation therapy in each of the four chambers of the heart, the atrial and ventricular pulse generators, 890 and 892, may include dedicated, independent pulse generators, multiplexed pulse generators, or shared pulse generators. The pulse generators, 890 and 892, are controlled by the microcontroller 880 via appropriate control signals, S1 and S2 respectively, to trigger or inhibit the stimulation pulses.

The microcontroller 880 further includes timing control circuitry 896 which is used to control pacing parameters (e.g., the timing of stimulation pulses) as well as to keep track of the timing of refractory periods, noise detection windows, evoked response windows, alert intervals, marker channel timing, etc., which is well known in the art. Examples of pacing parameters include, but are not limited to, atrio-ventricular delay, interventricular delay and interatrial delay.

The switch bank 894 includes a plurality of switches for connecting the desired electrodes to the appropriate I/O circuits, thereby providing complete electrode programmability. Accordingly, the switch 894, in response to a control signal S3 from the microcontroller 880, determines the polarity of the stimulation pulses (e.g., unipolar, bipolar, etc.) by selectively closing the appropriate combination of switches (not shown) as is known in the art.

Atrial sensing circuits 898 and ventricular sensing circuits 820 may also be selectively coupled to the right atrial lead 860, coronary sinus lead 870, and the right ventricular lead 850, through the switch 894 for detecting the presence of cardiac activity in each of the four chambers of the heart. Accordingly, the atrial (ATR. SENSE) and ventricular (VTR. SENSE) sensing circuits, 898 and 820, may include dedicated sense amplifiers, multiplexed amplifiers, or shared amplifiers. The switch 894 determines the “sensing polarity” of the cardiac signal by selectively closing the appropriate switches, as is also known in the art. In this way, the clinician may program the sensing polarity independent of the stimulation polarity.

Each sensing circuit, 898 and 820, preferably employs one or more low power, precision amplifiers with programmable gain and/or automatic gain control, bandpass filtering, and a threshold detection circuit, as known in the art, to selectively sense the cardiac signal of interest. The automatic gain control enables the device 810 to deal effectively with the difficult problem of sensing the low amplitude signal characteristics of atrial or ventricular fibrillation. Such sensing circuits, 898 and 820, can be used to determine cardiac performance values used in the present invention. Alternatively, an automatic sensitivity control circuit may be used to effectively deal with signals of varying amplitude.

The outputs of the atrial and ventricular sensing circuits, 898 and 820, are connected to the microcontroller 880 which, in turn, are able to trigger or inhibit the atrial and ventricular pulse generators, 890 and 892, respectively, in a demand fashion in response to the absence or presence of cardiac activity, in the appropriate chambers of the heart. The sensing circuits, 898 and 820, in turn, receive control signals over signal lines, S4 and S5, from the microcontroller 880 for purposes of measuring cardiac performance at appropriate times, and for controlling the gain, threshold, polarization charge removal circuitry (not shown), and timing of any blocking circuitry (not shown) coupled to the inputs of the sensing circuits, 898 and 820.

For arrhythmia detection, the device 810 includes an arrhythmia detector 882 that utilizes the atrial and ventricular sensing circuits, 898 and 820, to sense cardiac signals to determine whether a rhythm is physiologic or pathologic. The timing intervals between sensed events (e.g., P-waves, R-waves, and depolarization signals associated with fibrillation) can be classified by the microcontroller 880 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to assist with determining the type of remedial therapy that is needed (e.g., bradycardia pacing, anti-tachycardia pacing, cardioversion shocks or defibrillation shocks, collectively referred to as “tiered therapy”). Additionally, the arrhythmia detector 882 can perform arrhythmia discrimination, including tachyarrhythmia classification. The arrhythmia detector 882 can be implemented within the microcontroller 880, as shown in FIG. 8B. Thus, this detector 882 can be implemented by software, firmware, or combinations thereof. It is also possible that all, or portions, of the arrhythmia detector 882 can be implemented using hardware. Further, it is also possible that all, or portions, of the arrhythmia detector 882 can be implemented separate from the microcontroller 880.

In accordance with embodiments of the present invention, the implant device 810 includes an arrhythmic event monitor 884, which can detect noise events using the techniques described above with reference to FIGS. 3 and 4. The arrhythmic event monitor 884, can be implemented within the microcontroller 880, as shown in FIG. 8B, and can be implemented using software, firmware, or combinations thereof. It is also possible for all, or portions, of the arrhythmic event monitor 884 to be implemented using hardware. Further, it is also possible for all, or portions, of the arrhythmic event monitor 884 to be implemented separate from the microcontroller 880.

The implantable device 810 can also include a pacing controller 886, which can adjust a pacing rate and/or pacing intervals based on measures of arterial blood pressure, in accordance with embodiments of the present invention. The pacing controller 886 can be implemented within the microcontroller 880, as shown in FIG. 8B. Thus, the pacing controller 886 can be implemented by software, firmware, or combinations thereof. It is also possible that all, or portions, of the pacing controller 886 can be implemented using hardware. Further, it is also possible that all, or portions, of the pacing controller 886 can be implemented separate from the microcontroller 880.

Still referring to FIG. 8B, cardiac signals are also applied to the inputs of an analog-to-digital (A/D) data acquisition system 822. The data acquisition system 822 is configured to acquire IEGM and/or ECG signals, convert the raw analog data into a digital signal, and store the digital signals for later processing and/or telemetric transmission to an external device 830. The data acquisition system 822 can be coupled to the right atrial lead 860, the coronary sinus lead 870, and the right ventricular lead 850 through the switch 894 to sample cardiac signals across any pair of desired electrodes. The data acquisition system 822 can be used to perform the bandpass filtering used in embodiments of the present invention. Alternatively, one or more other filters can be used.

The data acquisition system 822 can be coupled to the microcontroller 880, or other detection circuitry, for detecting an evoked response from the heart in response to an applied stimulus, thereby aiding in the detection of “capture”. Capture occurs when an electrical stimulus applied to the heart is of sufficient energy to depolarize the cardiac tissue, thereby causing the heart muscle to contract. The microcontroller 880 detects a depolarization signal during a window following a stimulation pulse, the presence of which indicates that capture has occurred. The microcontroller 880 enables capture detection by triggering the ventricular pulse generator 892 to generate a stimulation pulse, starting a capture detection window using the timing control circuitry 896 within the microcontroller 880, and enabling the data acquisition system 822 via control signal S6 to sample the cardiac signal that falls in the capture detection window and, based on the amplitude, determines if capture has occurred.

The implementation of capture detection circuitry and algorithms are well known. See for example, U.S. Pat. No. 4,729,376 (Decote, Jr.); U.S. Pat. No. 4,708,142 (Decote, Jr.); U.S. Pat. No. 4,686,988 (Sholder); U.S. Pat. No. 4,969,467 (Callaghan et. al.); and U.S. Pat. No. 5,350,410 (Mann et. al.), which patents are hereby incorporated herein by reference. The type of capture detection system used is not critical to the present invention.

The microcontroller 880 is further coupled to the memory 824 by a suitable data/address bus 826, wherein the programmable operating parameters used by the microcontroller 880 are stored and modified, as required, in order to customize the operation of the implantable device 810 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart within each respective tier of therapy. The memory 824 can also store data about blood perfusion and/or blood volume in an organ of interest.

The operating parameters of the implantable device 810 may be non-invasively programmed into the memory 824 through a telemetry circuit 828 in telemetric communication with an external device 830, such as a programmer, transtelephonic transceiver, or a diagnostic system analyzer. The telemetry circuit 828 can be activated by the microcontroller 880 by a control signal S7. The telemetry circuit 828 advantageously allows intracardiac electrograms and status information relating to the operation of the device 810 (as contained in the microcontroller 880 or memory 824) to be sent to the external device 830 through an established communication link S8. The telemetry circuit 828 can also be use to transmit arrhythmic and noise data to the external device 830. Optionally, the implant device 810 can further include a patient alert 838 that can indicate heart, and/or other organ dysfunction. The patient alert 838 receives a signal S11 from the controller 880 when predefined conditions are met.

For examples of telemetry devices, see U.S. Pat. No. 4,809,697, entitled “Interactive Programming and Diagnostic System for use with Implantable Pacemaker” (Causey, III et al.); U.S. Pat. No. 4,944,299, entitled “High Speed Digital Telemetry System for Implantable Device” (Silvian); and U.S. Pat. No. 6,275,734 entitled “Efficient Generation of Sensing Signals in an Implantable Medical Device such as a Pacemaker or ICD” (McClure et al.), which patents are hereby incorporated herein by reference.

The implantable device 810 additionally includes a battery 832 which provides operating power to all of the circuits shown in FIG. 8B. If the implantable device 810 also employs shocking therapy, the battery 832 should be capable of operating at low current drains for long periods of time, and then be capable of providing high-current pulses (for capacitor charging) when the patient requires a shock pulse. The battery 832 should also have a predictable discharge characteristic so that elective replacement time can be detected.

The implantable device 810 can also include a magnet detection circuitry (not shown), coupled to the microcontroller 880. It is the purpose of the magnet detection circuitry to detect when a magnet is placed over the implantable device 810, which magnet may be used by a clinician to perform various test functions of the implantable device 810 and/or to signal the microcontroller 880 that the external programmer 830 is in place to receive or transmit data to the microcontroller 880 through the telemetry circuits 828.

As further shown in FIG. 8B, the implant device 810 is also shown as having an impedance measuring circuit 834 which is enabled by the microcontroller 880 via a control signal S9. The known uses for an impedance measuring circuit 834 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds and heart failure condition; detecting when the device has been implanted; measuring stroke volume; and detecting the opening of heart valves, etc. The impedance measuring circuit 834 is advantageously coupled to the switch 894 so that any desired electrode may be used. The impedance measuring circuit 834 is not critical to the present invention and is shown only for completeness.

In the case where the implant device 810 is also intended to operate as an implantable cardioverter/defibrillator (ICD) device, it should detect the occurrence of an arrhythmia, and automatically apply an appropriate electrical shock therapy to the heart aimed at terminating the detected arrhythmia. To this end, the microcontroller 880 further controls a shocking circuit 836 by way of a control signal S10. The shocking circuit 836 generates shocking pulses of low (up to 0.5 Joules), moderate (0.5-10 Joules), or high energy (11 to 40 Joules), as controlled by the microcontroller 880. Such shocking pulses are applied to the patient's heart through at least two shocking electrodes, and as shown in this embodiment, selected from the left atrial coil electrode 876, the RV coil electrode 856, and/or the SVC coil electrode 858. As noted above, the housing 840 may act as an active electrode in combination with the RV electrode 856, or as part of a split electrical vector using the SVC coil electrode 858 or the left atrial coil electrode 876 (i.e., using the RV electrode as a common electrode).

The above described implantable device 810 was described as an exemplary pacing device. One or ordinary skill in the art would understand that embodiments of the present invention can be used with alternative types of implantable devices. Accordingly, embodiments of the present invention should not be limited to use only with the above described device.

The embodiments of the present invention have been described above with the aid of functional building blocks illustrating the performance of specified functions and relationships thereof. The boundaries of these functional building blocks have often been arbitrarily defined herein for the convenience of the description. Alternate boundaries can be defined so long as the specified functions and relationships thereof are appropriately performed. Any such alternate boundaries are thus within the scope and spirit of the claimed invention. For example, it would be possible to combine or separate some of the steps shown in FIGS. without substantially changing the overall events and results.

The previous description of the preferred embodiments is provided to enable any person skilled in the art to make or use the embodiments of the present invention. While the invention has been particularly shown and described with reference to preferred embodiments thereof, it will be understood by those skilled in the art that various changes in form and details may be made therein without departing from the spirit and scope of the invention.

What is claimed is:

1. A method of noise detection and response for use when monitoring for arrhythmias, comprising:
(a) using at least two electrodes to obtain a signal indicative of cardiac electrical activity;
(b) bandpass filtering the signal to obtain a filtered signal;
(c) monitoring for ventricular depolarizations based on comparisons of the filtered signal to a first threshold;
(d) monitoring for arrhythmias based on ventricular depolarization detections that occur as a result of the monitoring at step (c);
(e) monitoring for noise during one or more noise detection windows;
(f) determining, based on results of the monitoring for noise, when it is likely that the monitoring for arrhythmias is adversely affected by noise; and
(g) modifying whether and/or how the monitoring for arrhythmias is performed, when it is determined that the monitoring for arrhythmias is likely adversely affected by noise.

2. The method of claim 1, wherein steps (d) is performed before step (e), step (e) is performed before step (d), or step (d) and step (e) are performed simultaneously.

3. The method of claim 1, wherein the signal is one of an electrocardiogram (ECG) signal and an intracardiac electrogram (IEGM) signal.

4. The method of claim 3, wherein step (a) comprises:
using at least two electrodes implanted subcutaneously to obtain a subcutaneous ECG signal;
using at least two non-implanted surface electrodes to obtain a surface ECG signal; or
using at least two electrodes implanted within or on the heart to obtain an IEGM signal.

5. The method of claim 1, wherein step (b) comprises bandpass filtering the signal to achieve an effective frequency range of about 8.5 Hz to about 30 Hz.

6. The method of claim 1, wherein step (c) comprises determining a ventricular depolarization detection when the filtered signal crosses the first threshold.

7. The method of claim 1, wherein step (d) comprises monitoring for at least ventricular tachyarrhythmia (VT) and atrial fibrillation (AF).

8. The method of claim 1, wherein step (e) comprises monitoring for noise based on comparisons of portions of the filtered signal, corresponding to the noise detection windows, to a second threshold having a lower magnitude than the first threshold.

9. The method of claim 8, wherein step (e) includes determining that noise is detected within a noise detection window based on how many times the portion of the filtered signal, corresponding to the noise detection window, crosses the second threshold.

10. The method of claim 8, wherein the second threshold is a specified percentage of the first threshold, with the specified percentage having a lower limit of about 25% and an upper limit of about 85%.

11. The method of claim 8, wherein:
step (e) comprises monitoring for noise during a plurality of back-to-back noise detection windows; and
each said noise detection window comprises a length of time having a lower limit of about 90 ms and an upper limit of about 250 ms.

12. The method of claim 11, wherein each said noise detection window is about 125 ms.

13. The method of claim 11, wherein step (e) includes determining that noise is detected within a noise detection window if the portion of the filtered signal, corresponding to the noise detection window, crosses the second threshold at least N times, wherein N is an integer ≧1.

14. The method of claim 13, wherein N is an integer ≧4.

15. The method of claim 1, wherein step (f) comprises determining that it is likely that the monitoring for arrhythmias is adversely affected by noise, if noise detections occurred for a specified number of the immediately preceding ventricular depolarization windows.

16. The method of claim 15, wherein step (f) comprises determining that it is likely that the monitoring for arrhythmias is adversely affected by noise, if noise detections occurred for X out of the last Y immediately preceding ventricular depolarization windows, where Y is an integer ≧2, and X is an integer ≦Y.

17. The method of claim 1, wherein when it is determined that the monitoring for arrhythmias is likely adversely affected by noise, step (g) comprises one of the following:
inhibiting monitoring for arrhythmias;
ignoring arrhythmia detections; and
storing information about arrhythmia detections along with an indication that the arrhythmia detections occurred when it was determined that the monitoring for arrhythmias was likely adversely affected by noise.

18. The method of claim 1, wherein step (g) comprises entering a noise mode, when it is determined that the monitoring for arrhythmias is likely adversely affected by noise, wherein during the noise mode:
arrhythmias are continued to be monitored for at step (d); and
information about arrhythmia detections are stored along with at least one marker indicating that the arrhythmia detections occurred when the monitoring for arrhythmias was likely adversely affected by noise.

19. The method of claim 18, wherein the at least one marker comprises:
a noise entry marker; and
a noise exit marker.

20. The method of claim 1, wherein step (g) comprises entering an inhibit mode, when it is determined that the monitoring for arrhythmias is likely adversely affected by noise, wherein during the inhibit mode:
arrhythmias are not monitored for at step (d); or
arrhythmias are monitored for at step (d), but information about arrhythmia detections are not stored.

21. An implantable device, comprising:
one or more sensing circuits configured to obtain, using at least two electrodes, a signal indicative of cardiac electrical activity;
one or more filters configured to filter the signal to obtain a bandpass filtered signal;
one or more monitors, processors and/or controllers configured to
detect ventricular depolarizations based on comparisons of the bandpass filtered signal to a first threshold;
monitor for arrhythmias based on ventricular depolarization detections;
monitor for noise during one or more noise detection windows;
determine when it is likely that the arrhythmia monitoring is adversely affected by noise; and
modify whether and/or how the arrhythmias are to be monitored for, when it is determined that the arrhythmia monitoring is likely adversely affected by noise.

22. The implantable device of claim 21, wherein the controller is configured to monitor for arrhythmias and monitor for noise at different times and/or at the same time.

23. The implantable device of claim 21, wherein:
the implantable device comprises a cardiac monitoring device that does not have stimulation capabilities;
the at least two electrodes are configured to be implanted subcutaneously; and
the signal obtained by the one or more sensing circuits comprises a subcutaneous electrocardiogram.

24. The implantable device of claim 21, wherein:
the implantable device comprises a cardiac stimulation device configured to pace and/or shock a patient's heart;
the at least two electrodes are configured to be implanted within and/or on a patient's heart; and
the signal obtained by the one or more sensing circuits comprises an intracardiac electrogram.

25. The implantable device of claim 21, wherein when the one or more monitors, processors and/or controllers determine that arrhythmia monitoring is likely adversely affected by noise, the one or more monitors, processors and/or controllers are configured to:
inhibit monitoring for arrhythmias;
ignore arrhythmia detections; or store information about arrhythmia detections along with an indication that the arrhythmia detections occurred when it was determined that the arrhythmia monitoring was likely adversely affected by noise.

26. A non-implantable device, comprising:
one or more sensing circuits configured to obtain, using at least two surface electrodes, a signal indicative of cardiac electrical activity;
one or more filters configured to filter the signal to obtain a bandpass filtered signal;
one or more monitors, processors and/or controllers configured to
detect ventricular depolarizations based on comparisons of the bandpass filtered signal to a first threshold;
monitor for arrhythmias based on ventricular depolarization detections;
monitor for noise during one or more noise detection windows;
determine when it is likely that the arrhythmia monitoring is adversely affected by noise; and
modify whether and/or how the arrhythmias are to be monitored for, when it is determined that the arrhythmia monitoring is likely adversely affected by noise.

27. The non-implantable device of claim 26, wherein when the one or more monitors, processors and/or controllers determine that arrhythmia monitoring is likely adversely affected by noise, the one or more monitors, processors and/or controllers are configured to:
inhibit monitoring for arrhythmias;
ignore arrhythmia detections;
store information about arrhythmia detections along with an indication that the arrhythmia detections occurred when it was determined that the monitoring for arrhythmias was likely adversely affected by noise; or
display information about arrhythmia detections along with an indication that the arrhythmia detections occurred when it was determined that the monitoring for arrhythmias was likely adversely affected by noise.
